(19) United States
(12) Patent Application Publication
(10) Pub. No.: US 2008/0095734 A1
(43) Pub. Date: Apr. 24, 2008

(54) CHEMICAL FORMULATION AND PROCESS FOR DE-CUTICLEIZING HAIR STRANDS

(76) Inventor: Patty Young, Springfield, OH (US)

Correspondence Address: DINSMORE & SHOHL LLP, ONE DAYTON CENTRE, ONE SOUTH MAIN STREET, SUITE 1300, DAYTON, OH, 45402-2023, US

(21) Appl. No.: 11/877,014

(22) Filed: Oct. 23, 2007

Related U.S. Application Data
(60) Provisional application No. 60/862,505, filed on Oct. 23, 2006.

(51) Int. Cl. A61K 8/92; A61Q 5/00

(52) U.S. Cl. 424/74

(57) ABSTRACT

Embodiments of the present invention are directed to a chemical formulation for de-cuticleizing hair strands comprising boiled water, bleach, shampoo, conditioner, and botanical oil, wherein the shampoo comprises a silicon based composition.

### CROSS-REFERENCE TO RELATED APPLICATIONS

This application claims the benefit of U.S. Provisional Application Ser. No. 60/862,505, filed Oct. 23, 2006.

### TECHNICAL FIELD

Embodiments of the present invention are generally directed to methods and systems of de-cuticleizing hair, and are specifically directed to chemical formulations used to de-cuticleize the hair strand.

### SUMMARY

Over the past few decades, hair grafting and replacement has become a widespread practice. As would be familiar to one of ordinary skill in the art, hair replacement involves the placement or insertion of a human hair replacement unit onto a user's scalp via an adhesive, or other suitable means. This could include the unit being attached to other hair with bonding glue. It can also be attached with a weaving cord or a thin melted plastic applied with a glue gun. Furthermore, the hair unit may be braided into the hair with small tacks weaved in the hair. In other words, there are numerous methods for attaching hair to the scalp or to existing hair or hair extensions. Despite a natural look, the human hair strands may cause problems, especially problems due to cuticle expansion.

Referring to FIG. 1, a hair strand is shown having an expanded cuticle layer. The cuticle is an outer layer comprising overlapping, protective scale-like cells. The cuticle protects the inner portions of the hair strand, such as the medulla and cortex. If the cuticle is deprived of nutrients and/or moisture, it may become dried out and expand. By expanding, the cuticle extends outside the dimensions of the hair strand, and produces an unappealing look. One of ordinary skill in the art may call this cuticle expansion a “split end”. In addition to being unappealing, cuticle expansion adds costs, because it may require a user to get a new hair replacement unit, or it may require the user to get frequent touchups at a hair salon.

Accordingly, the present inventor has recognized the importance improved hair grafting systems and methods that overcome the problems of cuticle expansion.

According to one embodiment, a chemical formulation for de-cuticleizing hair strands is provided. The chemical formulation comprises boiled water, bleach, shampoo, conditioner, and botanical oil, wherein the shampoo comprises a silicon based composition.

According to another embodiment, a method of preparing this chemical formulation is provided. The method comprises boiling a solution of water, adding bleach, shampoo, conditioner, and botanical oil into the solution of water, wherein the shampoo comprises a silicon based composition, mixing the solution of water after the addition of bleach, shampoo, conditioner, and botanical oil, and producing the chemical formulation by boiling the mixed solution. The mixing step may occur for variable durations, for example, a period of less than about 10 seconds in one embodiment. Mixing periods of minutes or hours are also contemplated herein.

According to yet another embodiment, a method of de-cuticleizing hair strands using this chemical formulation is provided. The method comprises immersing a hair unit into the chemical formulation, and removing the hair unit from the chemical formulation. In further embodiments, the hair may be shampooed, conditioned, shaped, and styled upon application of the hair unit onto the customer's head.

These and additional features provided by the embodiments of the present invention will be more fully understood in view of the following detailed description, in conjunction with the drawings.

### BRIEF DESCRIPTION OF THE DRAWINGS

The following detailed description of specific embodiments of the present invention can be best understood when read in conjunction with the drawings enclosed herewith. The drawing sheets include:

FIG. 1 is a schematic view of a hair strand having expanded cuticles on its surface;

FIG. 2 is a schematic view of a hair strand after utilization of the present treatment method; and

FIG. 3 is a flow chart illustrating a method for de-cuticleizing hair according to one or more embodiments of the present invention.

The embodiments set forth in the drawings are illustrative in nature and not intended to be limiting of the invention defined by the claims. Moreover, individual features of the drawings and the invention will be more fully apparent and understood in view of the detailed description.

### DETAILED DESCRIPTION

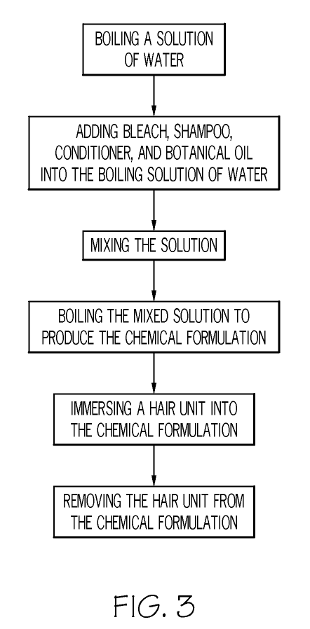

Referring generally to FIG. 3, the present methods and systems are directed to substantially eliminating cuticle expansion. According to this method, at least one hair unit must be provided. “Hair unit” as defined herein means a plurality of human hair strands. The method first requires a treatment basin or container in which to provide a treatment solution. The treatment solution first requires the addition of water, which is subsequently brought to a boil. After the water has boiled, numerous other chemical components are added. For example, and not by way of limitation, bleaches, oils, hair strengtheners, or shampoos are added into boiling solution. These products may include Ensure Conditioner by Nexxus and Botanical oil manufactured by Nexxus. It is preferable to use a shampoo comprising a silicon composition. To obtain the desired concentration, it is also contemplated to add additional water to the solution.

Referring again to FIG. 3, the components of the solution are subsequently mixed. The mixing process may be accelerated with heat or by stirring. The mixing times may vary depending on the amount of components added. In one exemplary embodiment, a mixing time is about 5 seconds or less. The amounts of each component added may vary based on the characteristics of the hair, e.g. dry or oily hair. In one exemplary embodiment, the prepared mixture may comprise 1 quart of water, ½ cup of bleach, 1 teaspoon of conditioner, and 1 tablespoon of botanical oil. The solution is then brought to a boil on a stove, a microwave, or via another suitable heating element.

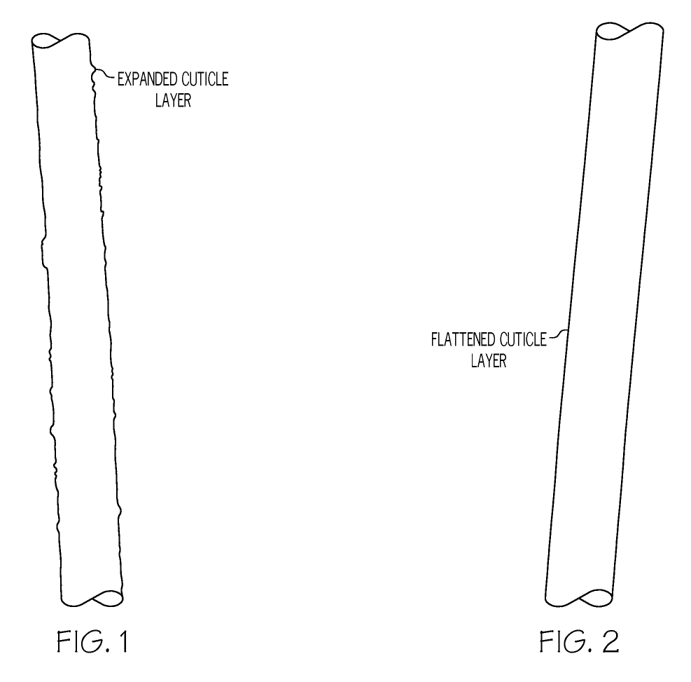

After the solution is prepared, the hair unit is immersed into the treatment solution. Although this discussion centers on the immersion of one hair unit, it is contemplated that multiple hair units may be treated at one time. The hair unit is immersed in the solution for less than about 30 seconds, and in a specific embodiment, less than about 5 seconds. Other immersion times may be desirable depending on the properties of the hair strands in the hair unit, for example, the oiliness of the hair, the thickness of the hair, the hair length, etc. During this immersion, the expanded cuticles in FIG. 1 are flattened to produce a more aesthetically pleasing hair strand as shown in FIG. 2. In FIG. 2, the cuticle layer of the hair strand is flattened or de-cuticleized, such that substantially no part of the cuticle extends outside the surface of the hair strand. In accordance with the present invention, the cuticle components extending outside the surface of the hair strand may be flattened or removed completely in the treatment solution.

Referring to FIG. 3, the hair unit is removed and, on some occasions, allowed to dry after immersion. On other occasions, after immersion, the hair is removed from the boiling solution, and is immediately rinsed, shampooed, and conditioned. To produce curly hair, the treated hair does not go through a drying process; however, some styles require drying and the time varies depending on the amount of hair thickness and style preferred. To expedite the drying process, a blow dryer, or other similar drying components, may be used.

After the drying stage, additional products may be supplied to the hair unit. For example, and not by way of limitation, hair strengtheners and leave-in conditioners may be applied to provide additional robustness to the hair unit, as well as making the hair unit more aromatic. Additional treatment or processing steps are contemplated as within the spirit and scope of the present invention.

It is noted that terms like “preferably,” “commonly,” and “typically” are not utilized herein to limit the scope of the claimed invention or to imply that certain features are critical, essential, or even important to the structure or function of the claimed invention. Rather, these terms are merely intended to highlight alternative or additional features that may or may not be utilized in a particular embodiment of the present invention.

For the purposes of describing and defining the present invention it is noted that the terms “substantially” and “about” are utilized herein to represent the inherent degree of uncertainty that may be attributed to any quantitative comparison, value, measurement, or other representation. These terms are also utilized herein to represent the degree by which a quantitative representation may vary from a stated reference without resulting in a change in the basic function of the subject matter at issue.

Having described the invention in detail and by reference to specific embodiments thereof, it will be apparent that modifications and variations are possible without departing from the scope of the invention defined in the appended claims. More specifically, although some aspects of the present invention are identified herein as preferred or particularly advantageous, it is contemplated that the present invention is not necessarily limited to these preferred aspects of the invention. For example, it is contemplated that the concepts of the present invention will be applicable to a variety of multi-projector systems.

What is claimed is:

1. A chemical formulation for de-cuticleizing hair strands comprising boiled water, bleach, shampoo, conditioner, and botanical oil, wherein the shampoo comprises a silicon based composition.

2. The chemical formulation of claim 1 further comprising hair strengtheners.

3. The chemical formulation of claim 1 wherein the chemical formulations comprises 1 quart of water, ½ cup of bleach, 1 teaspoon of conditioner, and 1 tablespoon of botanical oil.

4. A hair unit treated by the chemical formulation of claim 1.

5. A method of de-cuticleizing hair strands comprising:
providing the chemical formulation of claim 1;
immersing a hair unit into the chemical formulation;
removing the hair unit from the chemical formulation;

6. The method of claim 5 further comprising one or more of the steps of rinsing the hair unit, shampooing the hair unit, and conditioning the hair unit.

7. The method of claim 5 wherein the hair unit is immersed in the solution for less than about 10 seconds.

8. The method of claim 5 wherein the hair unit is immersed in the solution for less than about 5 seconds.

9. The method of claim 5 further comprising adding hair strengtheners or leave-in-conditioners after the drying step.

10. A method of preparing a chemical formulation for de-cuticleizing hair strands comprising the steps of:
boiling a solution of water;
adding bleach, shampoo, conditioner, and botanical oil into the solution of water, wherein the shampoo comprises a silicon based composition;
mixing the solution of water after the addition of bleach, shampoo, conditioner, and botanical oil for a period of up to 10 seconds; and
producing the chemical formulation by boiling the mixed solution.

11. The method of claim 10 wherein the solution is mixed for 5 seconds.

12. The method of claim 10 further comprising adjusting the concentration of the solution by adding water.
